(19) United States
(12) Patent Application Publication
(10) Pub. No.: US 2025/0135173 A1
(43) Pub. Date: May 1, 2025

(54) MICRONEEDLE, MICRONEEDLE PATCH AND METHOD OF MANUFACTURING A MICRONEEDLE

(71) Applicant: NATIONAL CHENG KUNG UNIVERSITY, Tainan City (TW)

(72) Inventors: Mei-Chin CHEN, Tainan City (TW); Ming-Thau SHEU, Taipei City (TW); Chih-Chi CHANG, Tainan City (TW); Chien-Chien YEH, Tainan City (TW)

(21) Appl. No.: 18/788,854

(22) Filed: Jul. 30, 2024

(30) Foreign Application Priority Data
Oct. 25, 2023 (TW) 112140826

(51) Int. Cl. A61M 37/00

(52) U.S. Cl. CPC A61M 37/0015; A61M 2037/0053

(57) ABSTRACT

A microneedle is provided in some embodiments of the present disclosure, including a supporting portion, a needle tip portion and an adhesion layer. The needle tip portion is disposed above the supporting portion, including an active substance. The adhesion layer is disposed between the supporting portion and the needle tip portion, including an adhesive material and a viscosity-reducing substance distributed in the adhesive material. A microneedle patch and a method are further provided in some embodiments of the present disclosure.

### CROSS-REFERENCE TO RELATED APPLICATION

This application claims priority to Taiwan Application Serial Number 112140826, filed Oct. 25, 2023, which is herein incorporated by reference.

### BACKGROUND

### Field of Invention

The present disclosure is related to a microneedle, microneedle patch and a method of manufacturing a microneedle.

### Description of Related Art

Skin aging is the most obvious external manifestation of human body aging. Whether it is endogenous aging of the skin caused by aging or exogenous aging caused by ultraviolet radiation, the content of collagen in the dermal layer of the skin will be reduced, causing the skin to shrink, form wrinkles, and lose elasticity. Commonly used clinical dermal fillers mainly include collagen or hyaluronic acid (HA), used as a temporary filler, and poly-L-lactic acid (PLLA), used as a semi-permanent filler. Since dermal fillers are mainly in the form of injections, they need to be performed by a doctor in person, and the results will vary depending on the doctor's operating skills.

Injection forms commonly suffer from problems such as blood stasis, hematoma, and pain due to the difficulty in controlling the injection location or injection dose (such as too deep or too much).

For example, clinically, the PLLA filler, Sculptra, usually causes skin bruising, redness, swelling, itching and pain after injection. It requires about a week of postoperative recovery period, and daily massage is required after the injection in order to make the PLLA microstructure evenly distributed in the skin and avoid uneven proliferation of collagen at the injection site. In addition, the PLLA used in Sculptra has irregular flake crystals with sharp corners, which will cause considerable stimulus to the skin tissue, causing inflammatory reactions to easily happen and lead to adverse symptoms including papules, nodules, foreign body granulomas, etc., at the injection site.

Therefore, how to provide an active substance (such as the aforementioned dermal filler) release dosage form that can reduce side effects, minimize operational risks, and improve the filling effect and the manufacture method of the active substance release dosage form is a problem that needs to be solved.

### SUMMARY

Some embodiments of the present disclosure provide a microneedle, inlcduing: a supporting portion, a needle tip portion and an adhesion layer. The needle tip portion is disposed above the supporting portion, including an active substance. The adhesion layer is disposed between the supporting portion and the needle tip portion, and the adhesion layer includes an adhesive material and a viscosity-reducing substance distributed in the adhesive material.

In some embodiments, the supporting portion includes a supporting portion material, and the supporting portion material includes a viscous substance, a non-viscous substance, or a combination thereof.

In some embodiments, the viscous substance includes hyaluronic acid (HA), sodium polyglutamate, polyvinyl alcohol (PVA), polyvinylpyrrolidone (PVP), carboxymethyl cellulose (CMC), gelatin, or a combination thereof.

In some embodiments, the non-viscous substance includes chitosan, polylactic acid (PLA), polyglycolic acid (PGA), polycaprolactone (PCL), polyetheretherketone (PEEK), poly(lactic-co-glycolic acid) (PLGA), polymethylmethacrylate (PMMA), polyethylene, or a combination thereof.

In some embodiments, the adhesive material includes hyaluronic acid, polyethylene glycol, polyoxyethylene, polyvinylpyrrolidone, polyvinyl alcohol, polyglutamic acid, gelatin, water soluble sugar, or a combination thereof.

In some embodiments, the viscosity-reducing substance is hydrophilic solubilizer.

In some embodiments, the viscosity-reducing substance includes sucrose, lactose, mannitol, mannose, galactose, glucose, fructose, maltose, trehalose, polyethylene glycol, or a combination thereof.

In some embodiments, a weight percentage of the viscosity-reducing substance in the adhesion layer is from 5% to 50% based on 100% by a total weight of the adhesion layer.

In some embodiments, a weight percentage of the viscosity-reducing substance in the adhesion layer is from 5% to 25% based on 100% by a total weight of the adhesion layer.

In some embodiments, the needle tip portion further includes a biodegradable material, and the biodegradable material includes hyaluronic acid, polyvinylpyrrolidone, polyvinyl alcohol, polyglutamic acid, starch, gelatin, chitosan, carboxymethyl cellulose, or a combination thereof.

In some embodiments, the active substance includes hyaluronic acid, polylactic acid (PLA), polyglycolic acid, poly(lactic-co-glycolic acid) (PLGA), polycaprolactone (PCL), calcium hydroxyapatite (CaHA), collagen, carboxymethyl cellulose, bioceramic, or a combination thereof.

In some embodiments, the active substance is represented as a microsphere.

Some embodiments of the present disclosure provide a microneedle patch, including: the abovementioned microneedle and a patch fixed on the supporting portion of the microneedle.

Some embodiments of the present disclosure provides a method of manufacturing a microneedle, including: mixing a needle tip portion material, an active substance and water to obtain a needle tip portion solution; filling the needle tip portion solution in a microneedle mold to obtain a shaped needle tip portion solution; drying the shaped needle tip portion solution to obtain a needle tip portion; mixing an adhesive material, a viscosity-reducing substance and water to obtain an adhesion layer solution; filling the adhesion layer solution in the microneedle mold which retains the needle tip portion to cover the needle tip portion with the adhesion layer solution and obtain a shaped adhesion layer solution; providing a supporting portion; placing the supporting portion in the microneedle mold which retains the needle tip portion and the shaped adhesion layer solution to make the shaped adhesion layer solution adhere to the supporting portion and the needle tip portion; and drying the shaped adhesion layer solution to obtain the microneedle.

In some embodiments, a weight percentage of the needle tip portion material in the needle tip portion solution is from 3% to 50% based on 100% by a total weight of the needle tip portion solution.

In some embodiments, the needle tip portion material includes a biodegradable material.

In some embodiments, the step of filling the needle tip portion solution in the microneedle mold includes: covering the microneedle mold with the needle tip portion solution; and pressing the needle tip portion solution first time using a first needle tip portion pressing device to allow the needle tip portion solution to fill up the microneedle mold and obtain the shaped needle tip portion solution. The step of drying the shaped needle tip portion solution includes: drying the shaped needle tip portion solution first time to allow the shaped needle tip portion solution to be initially solidified; and pressing the shaped needle tip portion solution second time using a second needle tip portion pressing device to allow the shaped needle tip portion solution which is initially solidified to be further shaped; and drying the shaped needle tip portion solution which is further shaped second time to obtain the needle tip portion.

In some embodiments, a weight percentage of the adhesive material in the adhesion layer solution is from 3% to 60%, and a weight percentage of the viscosity-reducing substance in the adhesion layer solution is from 0.15% to 30% based on 100% by a total weight of the adhesion layer solution.

In some embodiments, a weight percentage of the viscosity-reducing substance in the adhesion layer solution is from 3% to 15% based on 100% by a total weight of the adhesion layer solution.

In some embodiments, the viscosity-reducing substance includes sucrose, lactose, mannitol, mannose, galactose, glucose, fructose, maltose, trehalose, polyethylene glycol, or a combination thereof.

In some embodiments, the step of filling the adhesion layer solution in the microneedle mold which retains the needle tip portion includes pressing the adhesion layer solution using an adhesion layer pressing device to allow shaping the adhesion layer solution to cover the needle tip portion.

In some embodiments, the step of providing the supporting portion includes: mixing a supporting portion material with water to obtain a supporting portion solution; filling the supporting portion solution in a supporting portion mold to obtain a shaped supporting portion solution; and drying the shaped supporting portion solution to obtain the supporting portion.

In some embodiments, the step of filling the supporting portion solution in the supporting portion mold includes: covering the supporting portion mold with the supporting portion solution; and pressing the supporting portion solution first time using a first supporting portion pressing device to allow the supporting portion solution to fill up the supporting portion mold and obtain the shaped supporting portion solution; and the step of drying the shaped supporting portion solution includes: drying the shaped supporting portion solution to allow the shaped supporting portion solution to be initially solidified; pressing the shaped supporting portion solution second time using a second supporting portion pressing device to allow the shaped supporting portion solution which is initially solidified to be compacted on a bottom of the supporting portion mold; covering the supporting portion mold with the supporting portion solution again; pressing the supporting portion solution using the first supporting portion pressing device to allow the supporting portion solution which covers the supporting portion mold again to fill up the supporting portion mold and cover the shaped supporting portion solution which is compacted and obtain a secondary shaped supporting portion solution; and drying the secondary shaped supporting portion solution to allow the secondary shaped supporting portion solution to be shaped into the supporting portion.

In some embodiments, the step of providing the supporting portion, includes: disposing a supporting portion material on a supporting portion mold; heating the supporting portion material to allow the supporting portion material to be melted and fill up the supporting portion mold; and cooling the supporting portion material to obtain the supporting portion.

The microneedle of the present disclosure allows the needle tip portion containing a specific dose of the active substance to be left to slowly dissolve in a specific part of the skin as the adhesive material dissolves according to the design that the active substance is kept in the needle tip portion and the penetration depth of the needle tip portion is further extended by the supporting portion, thereby accurately controlling the release location and the release dose of the active substance. In addition, the release positions are dispersed since the active substance is dispersed to each puncture unit in the microneedle, which can reduce the risk of adverse symptoms caused by excessive concentrated release of the active substance.

Furthermore, when the microneedle is removed from the puncture position, the proportion of the needle tip portion sticking to the supporting portion can be reduced by adding the viscosity-reducing substance to the adhesion layer. Therefore, more needle tip portion can be retained in the puncture position, the amount that the active substance of the needle tip portion releases into the skin can be increased, the delivery efficiency can be increased, thereby enhancing the effect of stimulating collagen regeneration.

### BRIEF DESCRIPTION OF THE DRAWINGS

In order to allow the above-mentioned and other purposes, features, advantages and embodiments of the present disclosure to be more clearly understood, accompanying drawing is described as follows:

FIG. 1A illustrates a schematic diagram of a microneedle in some embodiments of the present disclosure.

FIG. 1B illustrates a schematic diagram of a microneedle patch in some embodiments of the present disclosure.

FIG. 2 illustrates a flow chart of a method of manufacturing a microneedle in some embodiments of the present disclosure.

FIG. 3A illustrates a microneedle manufactured by Example 1 of the present disclosure (part A and part B), and the results of puncture test after puncturing the porcine skin with the microneedle and then removing the supporting portion from the porcine skin (part C represents the supporting portion after removal, and part D represents the surface of porcine skin).

FIG. 3B illustrates the results of the puncture tests using two kinds of microneedles with an adhesion layer respectively including viscosity-reducing substance or including no viscosity-reducing substance to puncture porcine skin. Part A and part B show the results of the puncture tests in which viscosity-reducing substance (trehalose) is not included in the adhesion layer. Part A shows the supporting portion after removal, and part B shows the surface of the porcine skin. Part C and part D show the results of the puncture tests in which viscosity-reducing substance (trehalose) is included in the adhesion layer. Part C is the supporting portion after removal, and part D is the surface of the porcine skin. Part C and part D of FIG. 3B are part C and part D of FIG. 3A.

FIG. 4 illustrates the observation of the release of fluorescently labeled PLLA microspheres (PLLA MPs) in the skin tissue sections after puncturing porcine skin or rat skin with the microneedle of Example 1.

FIG. 5 illustrates the release of PLLA MPs at the puncture position that are detected via fluorescence at different time points after puncturing rat skin with the microneedle of Example 1.

FIG. 6 and FIG. 7 illustrate the immunohistochemical staining pictures of the microneedle puncture area or the intradermal injection area at week 4 after puncturing or intradermally injecting the rat skin, in which FIG. 6 is the result of Hematoxylin & Eosin stain (H&E stain), and FIG. 7 is the result of Masson's trichrome stain. In FIG. 6 and FIG. 7, the group of intradermal injection of phosphate buffered saline (PBS) is referred to as PBS ID group, the group of puncture with the microneedle that the needle tip portion is hyaluronic acid (PLLA MPs are not included) is referred to as HA MNs group, the group of intradermal injection of PLLA MPs is referred to as PLLA MPs ID group, and the group of puncture with the microneedle of Example 1 is referred to as PLLA MP-MNs group.

### DETAILED DESCRIPTION

It is to be understood that different implementations or embodiments provided in the following may implement different features of the subject matter of the present disclosure. The embodiments of specific components and arrangements are used to simplify the disclosure and not to limit the disclosure. Of course, these are only examples and are not intended to be limiting. For example, the description below that the first feature is formed on the second feature includes the two being in direct contact, or there are other additional features between the two that are not in direct contact. Furthermore, the present disclosure may repeat reference numerals and/or symbols in the various embodiments. Such repetition is for simplicity and clarity and does not represent a relationship between the various embodiments and/or configurations discussed.

As used herein, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, the term “about” means that the value of a given quantity varies within 5% of the value (for example, ±1%, ±2%, ±3%, ±4%, 5% of the value). These values are examples only and are not intended to be limiting. It should be understood that the term “about” may mean a percentage of the value of a given quantity as interpreted by one skilled in the relevant art in light of the teachings herein.

As used herein, unless otherwise defined (for example, with emphasis on “not including”), the terms “higher than”, “lower than”, “greater than”, “less than”, etc., are assumed to include the relative value they refer to, such as “higher than” 0.1 mg, which means 0.1 mg is included.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein can include a plurality of sub-steps or actions.

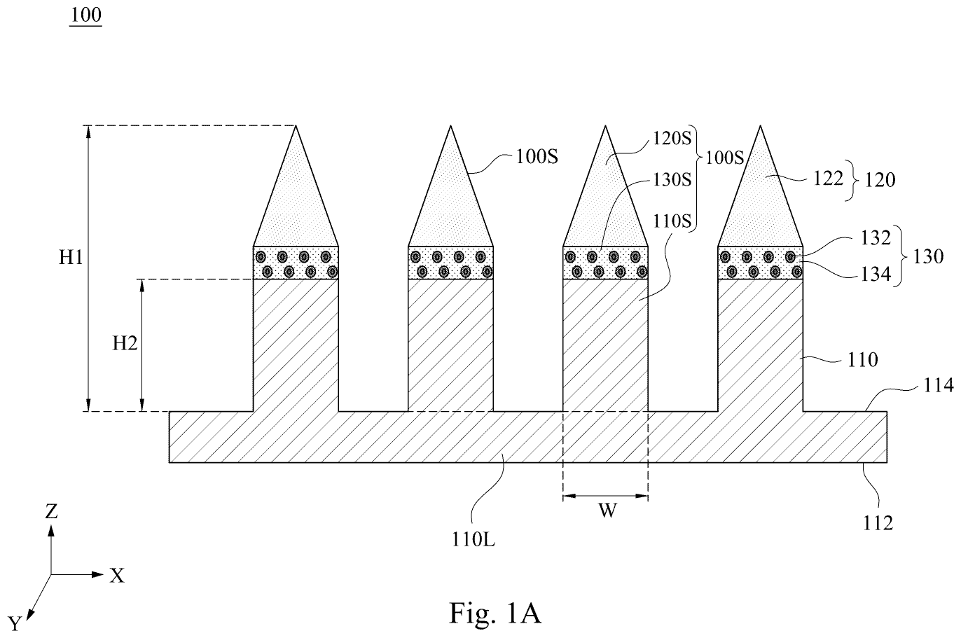

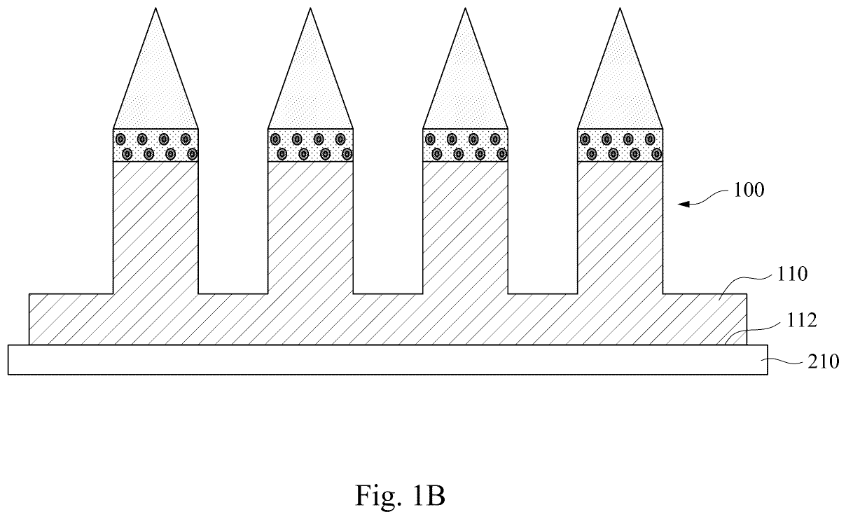

Please refer to FIG. 1A, some embodiments of the present disclosure provide a microneedle 100, including a supporting portion 110, a needle tip portion 120 and an adhesion layer 130. The needle tip portion 120 is disposed above the supporting portion 110, including an active substance 122. An adhesion layer 130 is disposed between the supporting portion 110 and the needle tip portion 120, and it includes an adhesive material 134 and a viscosity-reducing substance 132 distributed in the adhesive material 134.

The microneedle 100 of the present disclosure allows the needle tip portion 120 containing a specific dose of the active substance 122 to be left to slowly dissolve in a specific part of the skin as the adhesive material dissolves according to the design that the active substance 122 is kept in the needle tip portion 120 and the penetration depth of the needle tip portion 120 is further extended by the supporting portion 110. Therefore, the release location and the release dose of the active substance 122 are accurately controlled, the active substance 122 is dispersed, and the risk of adverse symptoms caused by excessive concentrated release of the active substance 122 is reduced. Since the release position and release dose are uniformly controlled through the structural design of the microneedle 100, the operating threshold can be lowered and the difference in subcutaneous filling effect caused by differences in the operator's operating techniques can be avoided.

In addition, when the microneedle 100 is removed from the puncture position, the proportion of the needle tip portion 120 sticking to the supporting portion 110 can be reduced by adding the viscosity-reducing substance 132 to the adhesion layer 130. Therefore, more needle tip portion 120 can be retained in the puncture position, the amount that the active substance 122 of the needle tip portion 120 releases into the skin can be increased, the delivery efficiency can be increased, thereby enhancing the effect of stimulating collagen regeneration.

In some embodiments, the supporting portion 110 includes a supporting portion material, and the supporting portion material includes viscous substance, non-viscous substance, or a combination thereof. In some embodiments, the viscous substance is water soluble substance, and the non-viscous substance is water-insoluble substance. The viscous substance can be dissolved in water or tissue fluid, for example, the solubility in each milliliter (mL) of water is higher than 0.1 mg. Relatively, the solubility of the non-viscous substance in each milliliter of water is lower (not including) than 0.1 mg. In some embodiments, the viscous substance includes hyaluronic acid, sodium polyglutamate, polyvinyl alcohol, polyvinylpyrrolidone, carboxymethyl cellulose, gelatin, or a combination thereof. In some embodiments, the non-viscous substance includes chitosan, polylactic acid, polyglycolic acid, polycaprolactone, polyetheretherketone, poly(lactic-co-glycolic acid) (PLGA), polymethylmethacrylate, polyethylene, or a combination thereof. It can be understood that using the non-viscous substance as the supporting portion material can minimize the situation in which the supporting portion 110 absorbs water vapor and becomes viscous in tissue fluid of the skin, softening the supporting portion110 and affecting the removal efficiency.

In some embodiments, the adhesive material 134 includes hyaluronic acid, polyethylene glycol, polyoxyethylene, polyvinylpyrrolidone, polyvinyl alcohol, polyglutamic acid, gelatin, water soluble sugar, or a combination thereof. In some embodiments, the viscosity-reducing substance 132 is hydrophilic solubilizer, for example, the viscosity-reducing substance 132 includes sucrose, lactose, mannitol, mannose, galactose, glucose, fructose, maltose, trehalose, polyethylene glycol, or a combination thereof. By adding the viscosity-reducing substance 132, the viscosity of the adhesion layer 130 adjacent to the supporting portion 110 can be adjusted to allow the supporting portion 110 to be separated from the needle tip portion 120 more easily. Therefore, the amount of the needle tip portion 120 adhering to the supporting portion110 is reduced, and the situation that the needle tip portion 120 is removed with the supporting portion110 together, or left on the surface of the skin when removing the supporting portion 110 are avoided.

In some embodiments, a weight percentage of the viscosity-reducing substance 132 in the adhesion layer 130 is from 5% to 50% based on 100% by a total weight of the adhesion layer 130, such as 5%, 10%, 15%, 20%, 30%, 40%, 50%, or a value in the aforementioned intervals, and a weight percentage of the adhesive material 134 is from 50% to 95%, such as 50%, 60%, 70%, 80%, 90%, 95%, or a value in the aforementioned intervals. If a weight percentage of the viscosity-reducing substance 132 is too high or a weight percentage of the adhesive material 134 is too low, the efficiency of the adhesion layer 130 on fixing the needle tip portion 120 and the supporting portion 110 is limited, and the probability of separation of the needle tip portion 120 from the supporting portion 110 is increased when taking the microneedle 100. If a weight percentage of the viscosity-reducing substance 132 is too low or a weight percentage of the adhesive material 134 is too high, the adhesion layer 130 is too sticky, and the probability that the needle tip portion 120 adheres to the supporting portion 110 is increased when the microneedle 100 is removed from the puncture position. In some embodiments, a weight percentage of the viscosity-reducing substance 132 in the adhesion layer 130 is from 5% to 25% based on 100% by a total weight of the adhesion layer 130.

In some embodiments, the active substance 122 of the needle tip portion 120 includes hyaluronic acid, polylactic acid (such as Poly-L-lactic Acid (PLLA) or Poly-D,L Lactic-Acid (PDLLA)), polyglycolic acid, poly(lactic-co-glycolic acid) (PLGA), polycaprolactone, calcium hydroxyapatite, collagen, carboxymethyl cellulose, bioceramic (biocompatible mineral crystals), or a combination thereof. People skilled in the art can select specific active substance 122 depending on demands. It can be understood that since the microneedle 100 can fix the depth of the puncture through regulation by the supporting portion 110 and control the appropriate amount of the active substance 122 to be released evenly from the needle tip portion120, the microneedle 100 can avoid serious foreign body inflammatory reaction caused by the excessive accumulation of released active substance 122, thereby avoiding adverse reactions such as papules, nodules, foreign body granulomas and other adverse symptoms, and having the better safety.

For example, polylactic acid can be used as dermal fillers to stimulate collagen regeneration. When polylactic acid is used as the active substance 122 of the needle tip portion 120, the micro-wound healing stimulation generated after punctured by the microneedle 100 can be combined with the characteristics of polylactic acid to induce collagen regeneration, and a two-pronged approach is provided to enhance the collagen regeneration effect. In addition, when collagen or hyaluronic acid, temporary fillers, is selected as the active substance 122, it will be quickly dissolved and absorbed by the skin after being punched into the skin. The wrinkle removal effect can only last for a few months to about a year and a half, limiting the maintenance time. Relatively, the active substance 122 extends the duration of the filling effect by selecting polylactic acid, which can be used as a semi-permanent filler.

In some embodiments, the active substance 122 is in the form of microspheres. Compared with the sharp edges of non-spherical forms (such as irregular shapes), the form of microspheres can reduce irritation to the skin and reduce the occurrence of adverse reactions.

In some embodiments, the needle tip portion 120 further includes a biodegradable material, and the biodegradable material includes hyaluronic acid, polyvinylpyrrolidone, polyvinyl alcohol, polyglutamic acid, starch, gelatin, chitosan, carboxymethyl cellulose, or a combination thereof. When the supporting portion 110 is removed from the puncture position, the needle tip portion 120 is retained in the skin, the biodegradable material can be dissolved in the skin, and the active substance 122 is released. In one embodiment, hyaluronic acid is selected as a biodegradable material to achieve good skin absorption effect, and hyaluronic acid can also accelerate wound recovery and shorten the recovery period.

In some embodiments, a weight percentage of the active substance 122 in the needle tip portion 120 is from 5% to 50% (such as 5%, 10%, 15%, 20%, 25%, 30%, 35%, 40%, 45%, 50%, or a value in the aforementioned intervals) based on 100% by a total weight of the needle tip portion 120, and a weight percentage of a biodegradable material in the needle tip portion 120 is from 50% to 95% (such as 50%, 60%, 70%, 80%, 90%, 95% or a value in the aforementioned intervals).

In some embodiments, the microneedle 100 has multiple puncture units 100S, each puncture unit 100S includes a needle tip portion unit 120S, an adhesion layer unit 130S and a supporting portion unit 110S, and each puncture unit 100S is linked with each other through a supporting portion linking unit 110L. In some embodiments, 1 cm² of microneedle 100 has 60 to 120 puncture units 100S, such as 60, 70, 80, 90, 100, 110, 120 or a value in the aforementioned intervals, and includes the active substance 122 ranging from 0.1 mg to 5 mg, such as 0.1 mg, 0.2 mg, 0.3 mg, 0.4 mg, 0.5 mg, 0.6 mg, 0.7 mg, 0.8 mg, 0.9 mg, 1 mg, 1.2 mg, 1.4 mg, 1.6 mg, 1.8 mg, 2 mg, 2.2 mg, 2.4 mg, 2.6 mg, 2.8 mg, 3 mg, 3.2 mg, 3.4 mg, 3.6 mg, 3.8 mg, 4 mg, 4.2 mg, 4.4 mg, 4.6 mg, 4.8 mg, 5 mg or a value in the aforementioned intervals, to achieve better collagen regeneration effect while avoiding adverse reactions caused by excessive active substance 122.

In some embodiments, a height H1 of the microneedle 100 (from a surface 114 of the supporting portion110 (or referred to as an upper surface of a base portion of the supporting portion 110) to an top of a needle tip portion 120) is from 300 μm to 1500 μm, such as 500 μm, 600 μm, 700 μm, 800 μm, 900 μm, 1000 μm, 1100 μm, 1200 μm, 1300 μm, 1400 μm, 1500 μm or a value in the aforementioned intervals. In some embodiments, a height H2 of the supporting portion 110 (from an interface between the supporting portion 110 and the adhesion layer 130 to a surface 114 of the supporting portion 110) is from 200 μm to 1200 μm, such as 200 μm, 300 μm, 400 μm, 500 μm, 600 μm, 700 μm, 800 μm, 900 μm, 1000 μm, 1100 μm, 1200 μm, or a value in the aforementioned intervals. If the height H1 or the height H2 is too short, the puncture position is too shallow, and the active substance 122 cannot be released in the dermal layer. If the height H1 or height H2 is too long, the punctured wound is too deep and the healing time is prolonged. In some embodiments, a width W that a basal surface of the puncture unit 100S (an interface between the supporting portion unit 110S and the supporting portion linking unit 110L) projects on the X-axis is about 200 μm to 500 μm, such as 200 μm, 300 μm, 400 μm, 500 μm, or a value in the aforementioned intervals. If the width W is too small, the supporting portion 110 has insufficient mechanical strength, leading to a higher risk of breakage. If the width W is too large, material costs will be increased.

Furthermore, please refer to FIG. 1B, some embodiments of the present disclosure provide a microneedle patch 200, including a microneedle100 and a patch 210 fixed on the supporting portion 110 of the microneedle 100. The patch 210 is fixed on the lower surface 112 of the supporting portion 110 and has viscosity. The patch 210 can adhere to the skin during puncture and fix the microneedle 100 in the skin to avoid wound expansion caused by the unexpected movement of the microneedle 100.

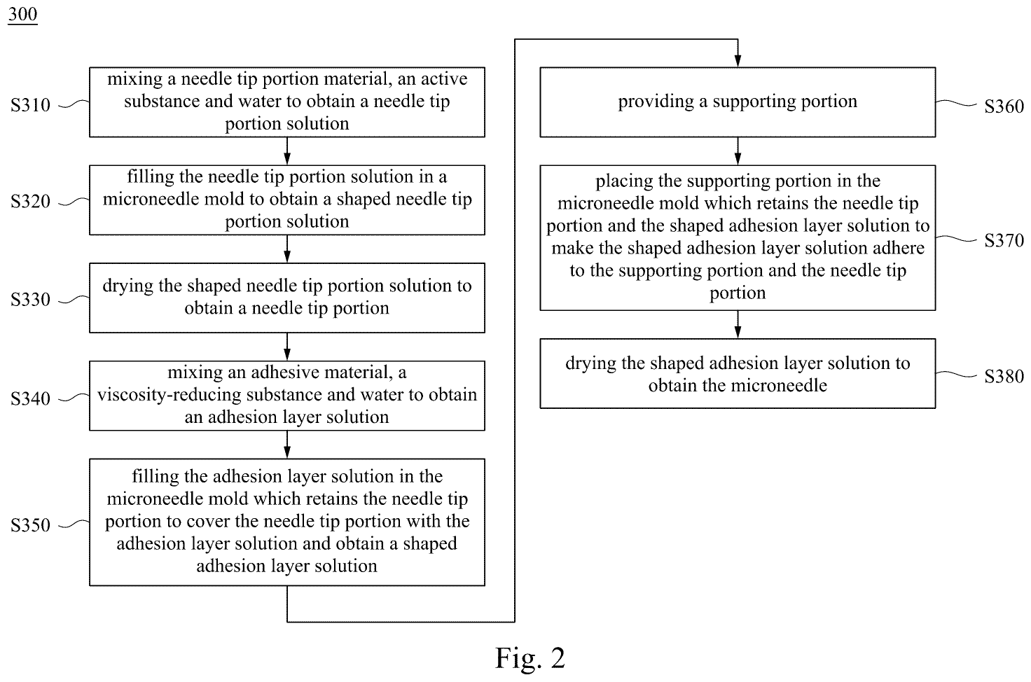

Next, please refer to FIG. 2 (combined with reference to FIG. 1A), some embodiments of the present disclosure provide a method 300 of manufacturing a microneedle 100.

First, step S310, a needle tip portion material, an active substance 122 and water are mixed to obtain a needle tip portion solution.

In some embodiments, the needle tip portion material includes a biodegradable material, such as hyaluronic acid, polyvinylpyrrolidone, polyvinyl alcohol, polyglutamic acid, starch, gelatin, chitosan, carboxymethyl cellulose, or a combination thereof. In some embodiments, a weight percentage of the needle tip portion material in the needle tip portion solution is from 3% to 50% based on 100% by a total weight of the needle tip portion solution, such as 3%, 5%, 10%, 15%, 20%, 25%, 30%, 35%, 40%, 45%, 50%, or a value in the aforementioned intervals. If the weight percentage is too low, the needle tip portion solution is too diluted, and the needle tip portion 120 is difficult to form and easily deformed. If the weight percentage is too high, the needle tip portion solution is too thick and difficult to operate.

In some embodiments, the active substance 122 includes hyaluronic acid, polylactic acid (such as poly-L-lactic acid (PLLA) or poly-D,L lactic-acid (PDLLA)), polyglycolic acid, poly(lactic-co-glycolic acid) (PLGA), polycaprolactone, calcium hydroxyapatite, collagen, carboxymethyl cellulose, bioceramic (biocompatible mineral crystals), or a combination thereof. In some embodiments, a weight percentage of the active substance 122 in a total weight of the needle tip portion solution is from 1% to 50%, such as 1%, 5%, 10%, 15%, 20%, 25%, 30%, 35%, 40%, 45%, 50%, or a value in the aforementioned intervals. In some embodiments, a weight percentage of the active substance 122 in a total weight of the needle tip portion solution is from 5 to 30%.

Step S320, the needle tip portion solution is filled in a microneedle mold to obtain a shaped needle tip portion solution.

In some embodiments, step S320 includes covering the microneedle mold with the needle tip portion solution; and pressing the needle tip portion solution first time using a first needle tip portion pressing device to allow the needle tip portion solution to fill up the microneedle mold and obtain the shaped needle tip portion solution. It can be understood that the needle tip portion solution may not fill well to the contours of the microneedle mold because of the high viscosity of the needle tip portion solution. It is ensured that the needle tip portion solution fills up the microneedle mold by initially pressing the needle tip portion solution, thereby improving the manufacturing yield of the needle tip portion 120. In some embodiments, a press part of the first needle tip portion pressing device is flat, facilitating the pushing of the needle tip portion solution into the microneedle mold.

Step S330, the shaped needle tip portion solution is dried to obtain a needle tip portion 120.

In some embodiments, the shaped needle tip portion solution is dried first time to allow the shaped needle tip portion solution to be initially solidified, and the shaped needle tip portion solution is pressed second time using a second needle tip portion pressing device to allow the shaped needle tip portion solution which is initially solidified to be further shaped, and the shaped needle tip portion solution which is further shaped second time is dried to obtain the needle tip portion 120. After initially solidified and then further shaped by the second needle tip portion pressing device, the needle tip portion solution can fit the internal contour of the microneedle mold better, and the manufacturing yield of the needle tip portion 120 can be improved. In some embodiments, the press part of the second needle tip portion pressing device is, for example, comb-like shape, which can be inserted deeply into the microneedle mold, so that the needle tip portion solution can better fit the contours inside the microneedle mold. In some embodiments, step S330 includes performing the first and second drying in an environment of 30° C. to 60° C. (such as 30° C., 40° C., 50° C., 60° C., or a value in the aforementioned intervals), where the time of the first drying is shorter than the time of the second drying.

Step S340, an adhesive material 134, a viscosity-reducing substance 132 and water are mixed to obtain an adhesion layer solution. The relevant descriptions of the adhesive material 134 and the viscosity-reducing substance 132 here can be found in the relevant recitation of FIG. 1A, and will not be described again here.

In some embodiments, a weight percentage of the adhesive material 134 in the adhesion layer solution is from 3% to 60% (such as 3%, 5%, 10%, 20%, 30%, 40%, 50%, 60%, or a value in the aforementioned intervals) based on 100% by a total weight of the adhesion layer solution, and a weight percentage of viscosity-reducing substance 132 in the adhesion layer solution is from 0.15% to 30% (such as 0.15%, 0.3%, 0.5%, 1%, 5%, 10%, 15%, 20%, 25%, 30%, or a value in the aforementioned intervals) based on 100% by the total weight of the adhesion layer solution. If the weight percentage of the adhesive material 134 is too high or a weight percentage of the viscosity-reducing substance 132 is too low, the adhesion layer solution will be too sticky and difficult to operate, which makes the adhesion layer 130 stick to the needle tip portion120 too strongly, which will cause the problem that the needle tip portion 120 adheres to the supporting portion 110 while removing the supporting portion 110 after puncturing the skin with microneedle 100. If a weight percentage of the adhesive material 134 is too low, or a weight percentage of the viscosity-reducing substance 132 is too high, the effect of the adhesion layer 130 on fixing the needle tip portion 120 and the supporting portion 110 is limited, and the probability of separation of the needle tip portion 120 from the supporting portion 110 increases while removing the microneedle 100. In some embodiments, a weight percentage of the viscosity-reducing substance 132 in the adhesion layer solution based on 100% by a total weight of the adhesion layer solution is from 3% to 15%.

Step S350, the adhesion layer solution is filled in the microneedle mold which retains the needle tip portion to cover the needle tip portion with the adhesion layer solution and obtain a shaped adhesion layer solution.

In some embodiments, step S350 includes pressing the adhesion layer solution using an adhesion layer pressing device to allow shaping the adhesion layer solution to cover the needle tip portion 120. In some embodiments, the adhesion layer pressing device and the first needle tip portion pressing device is the same.

Step S360, providing a supporting portion 110.

Depending on the difference in the supporting portion material, the supporting portion 110 can be prepared in different ways. For example, the following two preparation methods can be provided according to the difference in solubility of the supporting portion material in water.

When the supporting portion material is soluble in water or tissue fluid (e.g., with the solubility higher than 0.1 mg per milliliter of water), in some embodiments, step S360 includes mixing a supporting portion material with water to obtain a supporting portion solution; filling the supporting portion solution in a supporting portion mold to obtain a shaped supporting portion solution; and drying the shaped supporting portion solution to obtain the supporting portion 110.

In some embodiments, a weight percentage of a supporting portion material in the supporting portion solution based on 100% of a total weight of the supporting portion solution is from 30% to 70%, such as 30%, 40%, 50%, 60%, 70%, or a value in the aforementioned intervals. If the weight percentage is too low, the supporting portion solution is too diluted, the supporting portion 110 is difficult to form and easily deformed. If the weight percentage is too high, the supporting portion solution is too thick and difficult to operate.

In some embodiments, the step of filling the supporting portion solution in the supporting portion mold includes: covering the supporting portion mold with the supporting portion solution; and pressing the supporting portion solution first time using a first supporting portion pressing device to allow the supporting portion solution to fill up the supporting portion mold and obtain the shaped supporting portion solution. In some embodiments, a press part of the first supporting portion pressing device is flat, facilitating the pushing of the supporting portion solution into the supporting portion mold. In one embodiment, the first supporting portion pressing device and the first needle tip portion pressing device are the same.

In some embodiments, the step of drying the shaped supporting portion solution includes: drying the shaped supporting portion solution to allow the shaped supporting portion solution to be initially solidified; pressing the shaped supporting portion solution second time using a second supporting portion pressing device to allow the shaped supporting portion solution which is initially solidified to be compacted on a bottom of the supporting portion mold; covering the supporting portion mold with the supporting portion solution again; pressing the supporting portion solution using the first supporting portion pressing device to allow the supporting portion solution which covers the supporting portion mold again to fill up the supporting portion mold and cover the shaped supporting portion solution which is compacted so as to obtain a secondary shaped supporting portion solution; and drying the secondary shaped supporting portion solution to allow the secondary shaped supporting portion solution to be shaped into the supporting portion 110.

In some embodiments, the steps of drying the shaped supporting portion solution and drying the secondary shaped supporting portion solution include performing drying at an environment of 30° C. to 60° C. (such as 30° C., 40° C., 50° C., 60° C., or a value in the aforementioned intervals).

After initially solidified and then further shaped by the second supporting portion pressing device, the supporting portion solution can fit the internal contour of the supporting portion mold better, and the manufacturing yield of supporting portion 110 can be improved. In some embodiments, the press part of the second supporting portion pressing device is, for example, comb-like shape, which can be inserted deeply into the supporting portion mold, so that the supporting portion solution can better fit the contours inside the supporting portion mold. In some embodiments, the second supporting portion pressing device and the second needle tip portion pressing device are the same.

When the supporting portion material is hardly dissolved or insoluble in water or tissue fluid (such as solubility lower than (not including) 0.1 mg per milliliter of water, for example, polylactic acid), in some embodiments, step S360 includes disposing a supporting portion material on a supporting portion mold; heating the supporting portion material to allow the supporting portion material to be melted and fill up the supporting portion mold; and cooling the supporting portion material to obtain the supporting portion 110.

In some embodiments, heating the supporting portion material includes heating the supporting portion material at a temperature of 150° C. to 250° C. in a vacuum environment, such as 150° C., 160° C., 170° C., 180° C., 190° C., 200° C., 210° C., 220° C., 230° C., 240° C., 250° C., or a value in the aforementioned intervals, to ensure the supporting portion material is fully melted.

Step S370, the supporting portion 110 is placed in the microneedle mold which retains the needle tip portion 120 and the shaped adhesion layer solution to make the shaped adhesion layer solution adhere to the supporting portion 110 and the needle tip portion 120.

In some embodiments, step S370 includes applying force to the supporting portion 110 to push the supporting portion 110 into the microneedle mold, allowing the adhesion layer solution to joint the needle tip portion 120 and the supporting portion110.

Step S380, the shaped adhesion layer solution is dried to obtain the microneedle 100.

In some embodiments, the shaped adhesion layer solution is dried at an environment of from 30° C. to 40° C. (such as 30° C., 32° C., 34° C., 36° C., 38° C., 40° C., or a value in the aforementioned intervals) for 15 minutes to 45 minutes (such as 15 minutes, 20 minutes, 25 minutes, 30 minutes, 35 minutes, 40 minutes, 45 minutes, or a value in the aforementioned intervals).

In order to further illustrate the microneedle, the microneedle patch and the manufacture method of the microneedle provided by the various embodiments of the present disclosure, the following implementations are carried out. It should be noted that the following embodiments are only provided for demonstration purposes and are not limiting to the present disclosure.

### 1. Manufacture Method of Microneedle

### 1.1. Manufacture of Needle Tip Portion

First of all, the needle tip portion solution (including appropriate amount of PLLA microparticles (PLLA MPs), hyaluronic acid and water, where the weight percentage of hyaluronic acid in the needle tip portion solution is 33%) is spread on the microneedle mold. Then, force is applied on the needle tip portion solution using the first needle tip portion pressing device (a press part is flat) so that the needle tip portion solution fills up the microneedle mold to be shaped. The microneedle mold containing the needle tip portion solution is placed in an environment of about 37° C. for drying to allow the needle tip portion solution to be initially solidified. Furthermore, secondary force is performed on the needle tip portion solution using the second needle tip portion pressing device (its press part can go deep inside the microneedle mold), and the needle tip portion solution in the microneedle mold is pressed forward to the needle tip, making the needle tip portion solution be further shaped. Furthermore, the microneedle mold containing the needle tip portion solution is placed in an environment of about 37° C. to dry the needle tip portion solution for the second time, so that the needle tip portion solution is dried and solidified, and the needle tip portion contained in the microneedle mold is obtained.

### 1.2. Manufacture of Supporting Portion

Since the supporting portion material can be a viscous substance or a non-viscous substance, the following illustrates the manufacture method when the supporting portion material is a viscous substance or a non-viscous substance.

### 1.2.1 Supporting Portion Material is Non-Viscous Substance (Polylactic Acid is Taken as an Example)

The PLA thread is laid flat on the supporting portion mold, and heated at 200° C. for 3 hours in a vacuum that the pressure is 1 in.Hg, and then the pressure is released. This causes PLA thread to melt and fills up the supporting portion mold. After the polylactic acid thread cools to room temperature and solidifies into the supporting portion, the supporting portion is removed from the supporting portion mold so as to obtain the supporting portion.

### 1.2.2 Supporting Portion Material is Viscous Substance (Hyaluronic Acid is Taken as an Example)

The supporting portion solution (hyaluronic acid hydrogel formed by dissolving 50% of hyaluronic acid in water) is laid flat on the supporting portion mold (the internal contour of the supporting portion mold at least partially matches the contour near the open end of the microneedle mold), and force is applied on the supporting portion solution using the first supporting portion pressing device (its press part is flat) so that the supporting portion solution fills up the supporting portion mold. Then, the supporting portion mold containing the supporting portion solution is placed in an environment of about 37° C. for drying for several minutes to allow the supporting portion solution to be initially solidified. Furthermore, secondary force is performed on the supporting portion solution using the second supporting portion pressing device (its press part can go deep inside the supporting portion mold), and the supporting portion solution in the supporting portion mold is pressed forward to the bottom of the supporting portion mold. The supporting portion solution is laid flat on the supporting portion mold again, force is performed on the supporting portion solution for several seconds using the first supporting portion pressing device (its press part is flat) to make the supporting portion solution fill up the supporting portion mold and cover previously compacted supporting portion solution. Furthermore, the supporting portion mold is placed at room temperature overnight to make the supporting portion solution dry. Then, the supporting portion can be removed from the supporting portion mold so as to obtain the supporting portion.

### 1.3. Material Test of Adhesion Layer

The adhesion layer is used to connect the needle tip portion and the supporting portion. During the experiment, it was found that when the adhesion layer solution only contained hyaluronic acid, the needle tip portion was easily adhered to the supporting portion during the removal of the supporting portion, causing the loss of the needle tip portion and reducing the delivery efficiency of the active substance.

For avoiding the situation that the adhesion layer is left on the supporting portion during the removal of the supporting portion, different groups of the adhesion layer solutions (which form the adhesive layer after drying) are prepared by mixing different materials (hyaluronic acid+water, or water+hyaluronic acid+trehalose) in specific proportions, in which trehalose is used to replace a part of hyaluronic acid. The viscosity of each group (averaged after three repetitions) is compared under the condition that detection speed is 100% using a viscometer (Brookfield DVE digital viscometer, brand: Brookfield). The results are shown in Table 1.

TABLE 1

| | | Viscosity |
|---|---|---|
| Group | Formulation | (centipoise) |
| 1 | water:hyaluronic acid = 145:5 | 161 |
| 2 | water:hyaluronic | 131 |
| | acid:trehalose = 145:4.5:0.5 | |
| 3 | water:hyaluronic | 93 |
| | acid:trehalose = 145:4:1 | |

Table 1 represents that addition of trehalose can reduce the viscosity of the adhesion layer solution, and as the content of trehalose increases, the viscosity decreases. Viscosity of group 2 is lower than that of group 1, which can reduce the situation that the needle tip portion is left on the supporting portion during the removal of the supporting portion. However, compared with group 3, group 2 has better viscosity, providing better effect on fixing the needle tip portion and the supporting portion.

Therefore, a formula that contains both hyaluronic acid and trehalose with the ratio of hyaluronic acid to trehalose similar to that of Group 2 (hyaluronic acid:trehalose is 9:1) is used for the subsequent adhesion layer solution.

### 1.4. Manufacture of Microneedle

The adhesion layer solution (including trehalose with a weight percentage of 5% and hyaluronic acid with a weight percentage of 47%) is laid on the microneedle mold containing the needle tip portion mentioned in point 1.1. Force is applied on the adhesion layer solution using the adhesion layer pressing device to make the adhesion layer solution fill up the microneedle mold. Then, the supporting portion manufactured in point 1.2.1 is placed in the microneedle mold containing the needle tip portion and the adhesion layer solution to make the adhesion layer solution adhere to the supporting portion and the needle tip portion. Furthermore, the microneedle mold containing the needle tip portion, the adhesion layer solution and the supporting portion is placed at room temperature overnight, and then it is dried at 37° C. for 30 minutes. Subsequently, the microneedle is removed from the microneedle mold, and the follow-up performance tests are performed. It can be understood that regardless of whether the supporting portion uses viscous substance or non-viscous substance, the follow-up performance tests show a consistent trend. To simplify the context, the microneedle of Example 1 mentioned below is based on the microneedle whose supporting portion is non-viscous substance (polylactic acid).

The height of the microneedle in Example 1 is 1200 μm, and the height of the supporting portion is about 600 μm, which allows the needle tip portion to penetrate deep into the dermal layer of the skin. The base width of each puncture unit is approximately 300 μm, but is not limited to this. In principle, as long as the structural design can allow PLLA MPs to be released into the dermal layer, it should be included in the scope of the present disclosure. Furthermore, the microneedle of Example 1 has 81 puncture units per 1 cm² and contains 0.9 mg of the active substance, but it is not limited herein.

### 2. Performance Test of Microneedle

### 2.1. Puncture Ability of Microneedle (Visual Check)

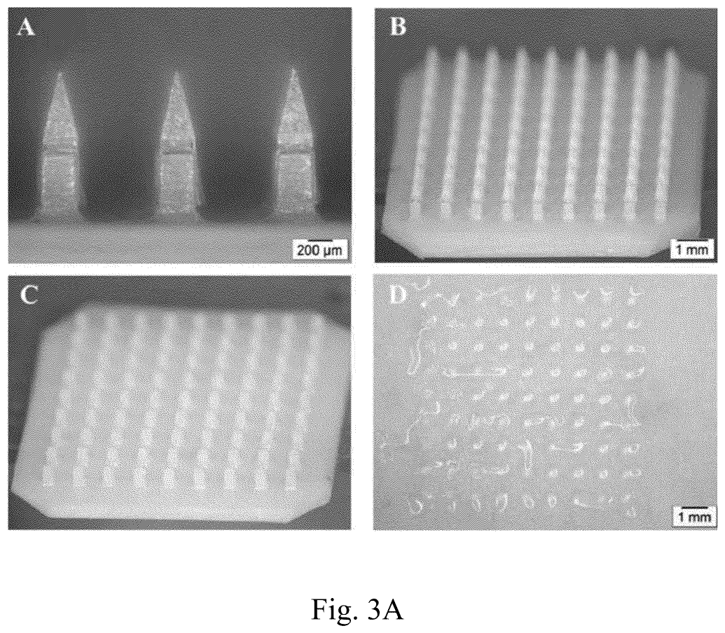

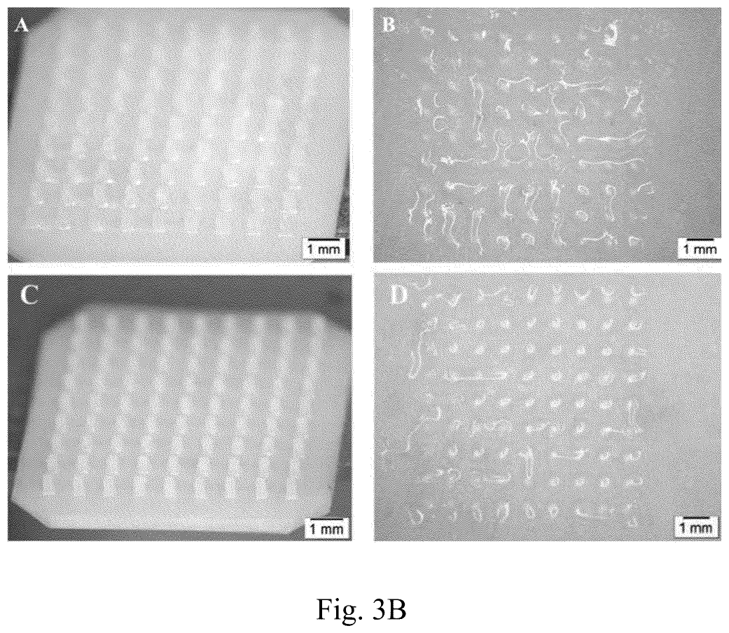

To test the puncture effect of the microneedle of Example 1 (adhesion layer includes trehalose distributed in hyaluronic acid), the microneedle of Example 1 (please refer to part A and part B of FIG. 3A) is fixed on the microneedle applicator, in which polylactic acid microspheres in the needle tip portion of the microneedle are labeled with Coumarin 6 fluorescence for use in observation. Furthermore, the microneedle is vertically applied into the porcine skin with a force of 7 Newtons by the microneedle applicator. The microneedle is attached to the porcine skin for 20 minutes. After the microneedle adhesion layer is dissolved, the patch is peeled off to remove the supporting portion.

For the results, please refer to part C (supporting portion) and part D (the surface of porcine skin) in FIG. 3A, respectively illustrating the situation of the removed supporting portion and the surface of porcine skin after removing the supporting portion of microneedle of Example 1 from porcine skin. Part C of FIG. 3A represents that the supporting portion can be completely removed from porcine skin. Fluorescent part of Part D of FIG. 3A represents that traces of fluorescent arrays can be observed on the surface of porcine skin after removing the supporting portion, indicating that the needle tip portion with fluorescent markings does have puncture ability and can be retained roughly intact in the porcine skin.

Relatively, if the adhesion layer of the microneedle only includes hyaluronic acid (the adhesion layer does not include trehalose, and the other manufacture methods of the microneedle are the same as Example 1), but not includes viscosity-reducing substance (such as trehalose), the needle tip portion will easily remain attached on the supporting portion during removal of the supporting portion, causing loss of the needle tip portion and reducing the delivery efficiency of the active substance. For example, please refer to FIG. 3B.

Part A and part B of FIG. 3B respectively illustrates the removed supporting portion and the surface of porcine skin after removing the supporting portion of microneedle from porcine skin when the adhesion layer of the microneedle only includes hyaluronic acid. As for part C and part D in FIG. 3B, they are the puncture results of the microneedle of Example 1 (the adhesion layer includes trehalose+hyaluronic acid), which are the same as part C and part D in FIG. 3A. For conveniently comparing the difference in results, they are juxtaposed with part A and part B of FIG. 3B.

To compare the removed supporting portion, part A and part C of FIG. 3B represent that after removing the microneedle, fluorescence residual amount of part C (the adhesion layer includes trehalose+hyaluronic acid) is reduced compared with that of part A (the adhesion layer includes hyaluronic acid), indicating that when the adhesion layer includes the viscosity-reducing substance (such as trehalose), the situation that the needle tip portion is left on the supporting portion during the removal of the supporting portion can be reduced to prevent the needle tip portion from being pulled out when the supporting portion is pulled out.

Compared with the surface of porcine skin, part D (the adhesion layer includes trehalose+hyaluronic acid) of FIG. 3B represents that the amount of fluorescent residue retaining on the surface of porcine skin is significantly lower than part B (the adhesion layer includes hyaluronic acid), indicating when the adhesion layer includes the viscosity-reducing substance (such as trehalose), the situation that the needle tip portion is pulled out and left on the surface of the porcine skin can be reduced. Therefore, the microneedle of Example 1 can improve delivery efficiency.

### 2.2. Release of Active Substance During Puncture

The skin of porcine and the skin of rat are punctured using the microneedle of Example 1 according to the test method similar to the puncture ability of the microneedle in Example 2.1. After the needle tip portion dissolves, the puncture positions are cut into appropriate sizes, embedded in cryo-embedding medium and then sliced to obtain skin tissue biopsy. Then, the puncture position of the microneedle (abbreviated as MN) and the distribution of fluorescently labeled PLLA MPs in the skin tissue biopsy are observed by fluorescence microscope.

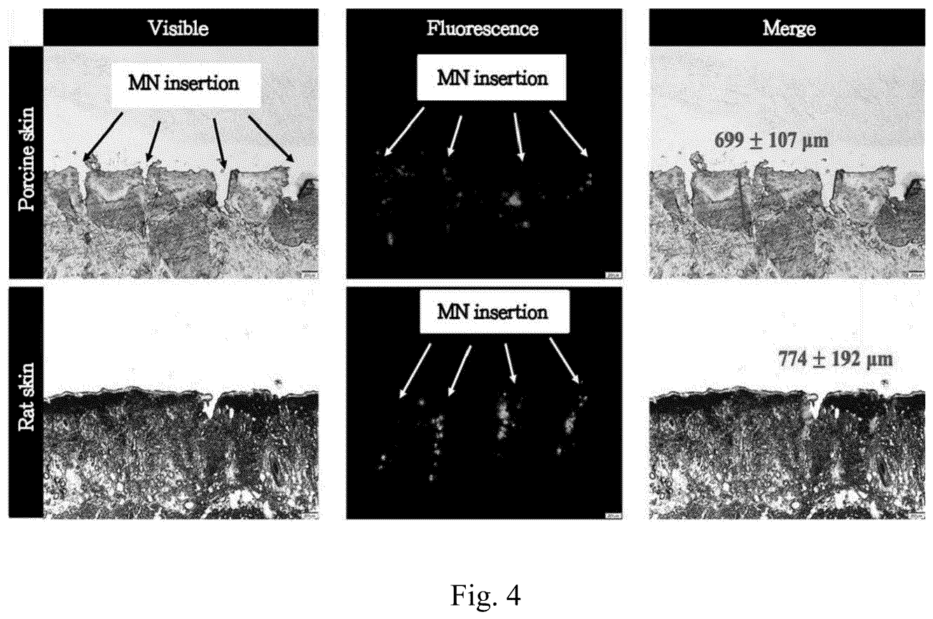

The results are shown in FIG. 4. It can be observed that whether it is porcine skin or rat skin, it can be successfully punctured by the microneedle. The depth is, for example, between 400 μm and 1000 μm, which can reach the dermal layer. Additionally, PLLA MPs are distributed along the puncture position, indicating that the active substance in the needle tip portion can be successfully released from the needle tip portion.

### 2.3. Distribution of Active Substance at Different Times

The microneedle is vertically applied into the rat skin with a force of 7 Newtons using an applicator. The microneedle is attached to the rat skin for 20 minutes. After the adhesion layer of the microneedle is dissolved by attaching the microneedle to the rat skin for 20 minutes, the medical breathable tape is peeled off to remove the supporting portion. At the time points of 0, 4 and 12 weeks, the rats are sacrificed, and the rat skin at the puncture position of the microneedle is removed, and the fluorescence of fluorescently labeled PLLA MPs in the rat skin is analyzed using confocal microscopy image system.

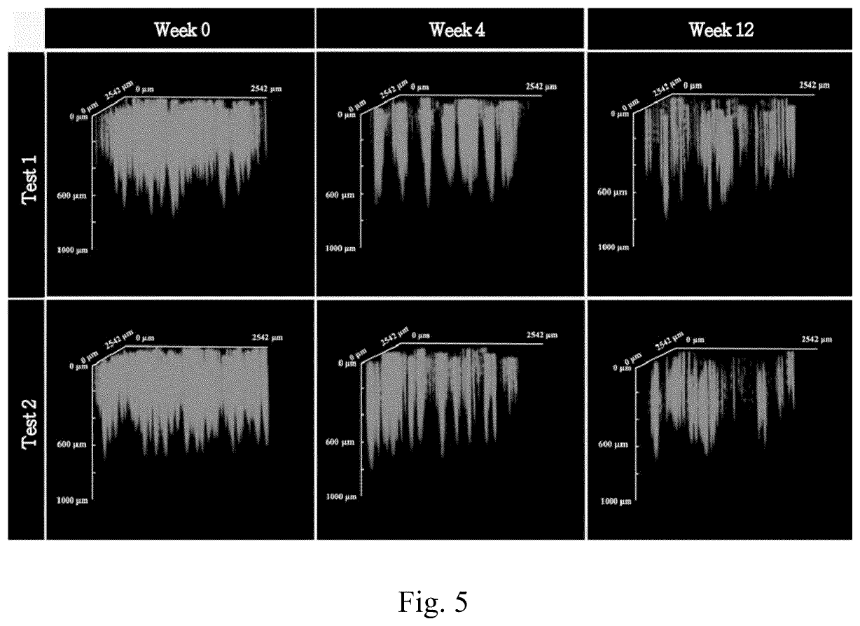

The results are shown in FIG. 5, it can be observed that PLLA MPs are densely distributed along the puncture position when they are just punctured. As time goes by, PLLA MPs gradually degrades, indicating that the PLLA MPs from the needle tip portion can be gradually absorbed by the skin after being punctured to the skin and released.

### 2.4. Distribution of Active Substance and Observation of Inflammatory Response

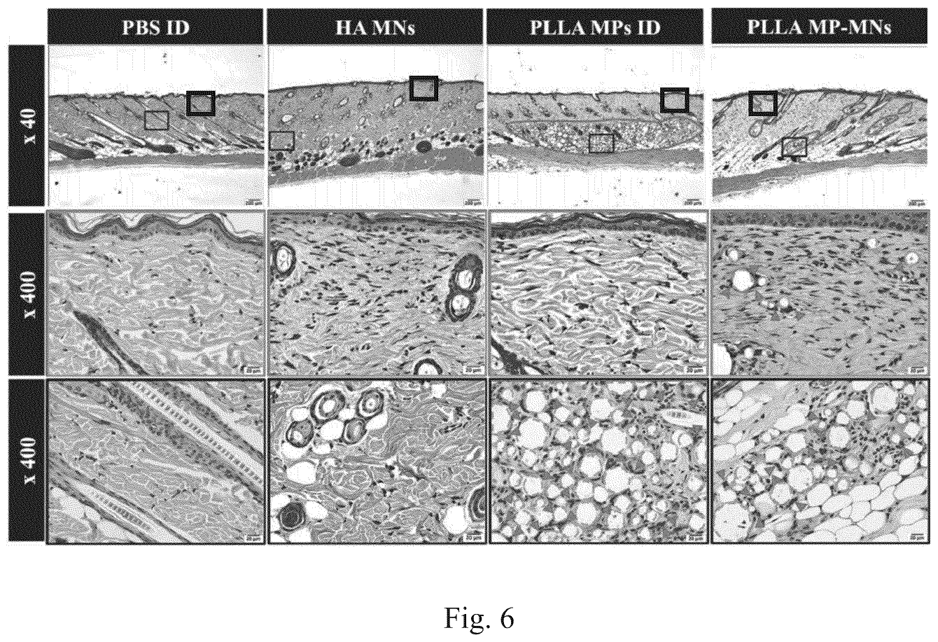

After puncturing the microneedle of Example 1 to the rats, the rats are sacrificed at the 4th week, the puncture area of the microneedle is marked and separated, and the rat skin tissue in this area is wrapped with lens paper to prevent it from being damaged during the subsequent fixation process. Furthermore, the rat skin tissue in lens paper is soaked in 10% neutral formalin. After fixing it for 23 hours, the rat skin tissue is taken out and washed by water. Then, the rat skin tissue is processed by dehydrated, embedded, and paraffin sectioned. Furthermore, the section is stained with Hematoxylin & Eosin stain (H&E stain), so that the cell nuclei is stained blue-purple, and the cytoplasm is stained pink, to observe whether the microneedle causes foreign body reactions (an increase in multinucleated giant cells) and inflammatory reactions. At the same time, in order to compare the differences in immune effects caused by different active substances and different dosage forms, the group of intradermal injection to the dermal layer (Intradermal injection, ID) and the group that the active substance is replaced with PBS or is only hyaluronic acid are served as controls during the process. The results are shown in FIG. 6, in which the groups that is intradermally injected with phosphate buffered saline (PBS) is referred to as PBS ID group, the group that is punctured by the microneedle of which the needle tip portion is hyaluronic acid (not including PLLA MP) is referred to as HA MNs group, the group that is intradermally injected with PLLA microspheres is referred to as PLLA MPs ID group, and the group that is punctured by the microneedle of Example 1 is referred to as PLLA MP-MNs group. In FIG. 6, the upper box in the first row is enlarged and displayed in the second row, representing the status of superficial dermis (skin depth is approximately less than 500 μm), and the lower box in the first row is enlarged and displayed in the third row, representing the status of deep dermis (skin depth is approximately more than 500 μm).

The results shown in FIG. 6 indicate that, PLLA MPs are indeed located in the dermis in the PLLA MP ID and PLLA MP-MNs groups (represented by the dotted line circle in the dermis). Additionally, multinucleated giant cell aggregation (marked by arrow) is observed in these two groups, suggesting that the administration of PLLA MPs into the skin elicits a more pronounced foreign body reaction.

Further, when comparing PLLA MPs ID group and PLLA MP-MNs group, it can be found that compared with the PLLA MPs ID group, PLLA MP-MNs group can alleviate the accumulation of multinucleated giant cells, reduce the occurrence of foreign body reaction and make the inflammatory response milder since PLLA MPs are more dispersed.

### 2.5. Formation of Collagen Fiber

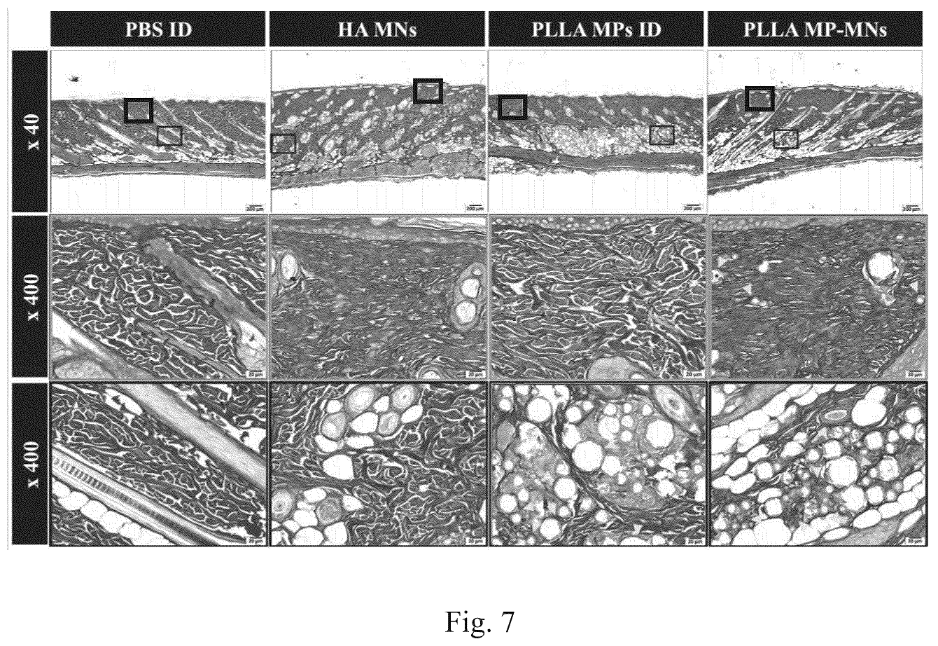

Following the steps and groups of Example 2.4, the sections of the puncture or injection site are determined by H&E staining in Example 2.4, and then the connective tissue was stained with Masson's trichrome stain to identify the location of the collagen. Please refer to FIG. 7 for the results. The upper box in the first row is enlarged and displayed in the second row, representing the status of superficial dermis (skin depth is approximately less than 500 μm), and the lower box in the first row is enlarged and displayed in the third row, representing the status of deep dermis (skin depth is approximately more than 500 μm).

The results in FIG. 7 show that when the temporary filler, hyaluronic acid, (HA MNs group) is applied, it can be seen that dense collagen structure is distributed in the upper layer of the dermis (superficial layer), and the tissue looks denser. In PLLA MPs ID group (intradermal injection), the newly generated collagen is distributed around the PLLA MPs in the lower dermis (deep layer). Furthermore, it can be seen that PLLA MP-MNs group has a synergistic effect brought by microneedle (MN) and PLLA MPs, and the dense collagen structure can be seen in both the upper and lower layers of the dermis. In addition, by comparing PLLA MPs ID group (intradermal injection) and PLLA MP-MNs group (microneedle puncture) whose active substances are both PLLA MPs, but using different injection methods, it can be found that the microneedle of the PLLA MP-MNs group can avoid excessive PLLA MPs aggregation, and at the same time, better stimulate surrounding skin cells and promote the formation of collagen.

In summary, compared with intradermal injection, PLLA MPs can be dispersed through skin puncture with the microneedle of Example 1, avoiding excessive aggregation of PLLA MPs, thereby avoiding too severe inflammatory reactions, and achieving better collagen regeneration effects.

Although the present invention has been disclosed in the above embodiments, it is not intended to limit the present invention. Anyone familiar with this technique can make various changes and modifications without departing from the spirit and scope of the present invention. The scope of protection of the invention shall be subjected to the scope of appended claims.

What is claimed is:

1. A microneedle, comprising:
a supporting portion;
a needle tip portion, disposed above the supporting portion, comprising an active substance; and
an adhesion layer, disposed between the supporting portion and the needle tip portion, comprising:
an adhesive material; and
a viscosity-reducing substance distributed in the adhesive material.

2. The microneedle of claim 1, wherein the supporting portion comprises a supporting portion material, and the supporting portion material comprises a viscous substance, a non-viscous substance, or a combination thereof.

3. The microneedle of claim 2, wherein the viscous substance comprises hyaluronic acid, sodium polyglutamate, polyvinyl alcohol, polyvinylpyrrolidone, carboxymethyl cellulose, gelatin, or a combination thereof.

4. The microneedle of claim 2, wherein the non-viscous substance comprises chitosan, polylactic acid, polyglycolic acid, polycaprolactone, polyetheretherketone, poly(lactic-co-glycolic acid), polymethylmethacrylate, polyethylene, or a combination thereof.

5. The microneedle of claim 1, wherein the adhesive material comprises hyaluronic acid, polyethylene glycol, polyoxyethylene, polyvinylpyrrolidone, polyvinyl alcohol, polyglutamic acid, gelatin, water soluble sugar, or a combination thereof.

6. The microneedle of claim 1, wherein the viscosity-reducing substance is hydrophilic solubilizer.

7. The microneedle of claim 6, wherein the viscosity-reducing substance comprises sucrose, lactose, mannitol, mannose, galactose, glucose, fructose, maltose, trehalose, polyethylene glycol, or a combination thereof.

8. The microneedle of claim 1, wherein a weight percentage of the viscosity-reducing substance in the adhesion layer is from 5% to 50% based on 100% by a total weight of the adhesion layer.

9. The microneedle of claim 8, wherein a weight percentage of the viscosity-reducing substance in the adhesion layer is from 5% to 25% based on 100% by a total weight of the adhesion layer.

10. The microneedle of claim 1, wherein the needle tip portion further comprises a biodegradable material, wherein the biodegradable material comprises hyaluronic acid, polyvinylpyrrolidone, polyvinyl alcohol, polyglutamic acid, starch, gelatin, chitosan, carboxymethyl cellulose, or a combination thereof.

11. The microneedle of claim 1, wherein the active substance comprises hyaluronic acid, polylactic acid, polyglycolic acid, poly(lactic-co-glycolic acid), polycaprolactone, calcium hydroxyapatite, collagen, carboxymethyl cellulose, bioceramic, or a combination thereof.

12. The microneedle of claim 1, wherein the active substance is represented as a microsphere.

13. A microneedle patch, comprising:
the microneedle of claim 1; and
a patch, fixed on the supporting portion of the microneedle.

14. A method of manufacturing a microneedle, comprising:
mixing a needle tip portion material, an active substance and water to obtain a needle tip portion solution;
filling the needle tip portion solution in a microneedle mold to obtain a shaped needle tip portion solution;
drying the shaped needle tip portion solution to obtain a needle tip portion;
mixing an adhesive material, a viscosity-reducing substance and water to obtain an adhesion layer solution;
filling the adhesion layer solution in the microneedle mold which retains the needle tip portion to cover the needle tip portion with the adhesion layer solution and obtain a shaped adhesion layer solution;
providing a supporting portion;
placing the supporting portion in the microneedle mold which retains the needle tip portion and the shaped adhesion layer solution to make the shaped adhesion layer solution adhere to the supporting portion and the needle tip portion; and
drying the shaped adhesion layer solution to obtain the microneedle.

15. The method of claim 14, wherein a weight percentage of the needle tip portion material in the needle tip portion solution is from 3% to 50% based on 100% by a total weight of the needle tip portion solution.

16. The method of claim 14, wherein the needle tip portion material comprises a biodegradable material.

17. The method of claim 14,
wherein the step of filling the needle tip portion solution in the microneedle mold comprises:
covering the microneedle mold with the needle tip portion solution; and
pressing the needle tip portion solution first time using a first needle tip portion pressing device to allow the needle tip portion solution to fill up the microneedle mold and obtain the shaped needle tip portion solution; and
wherein the step of drying the shaped needle tip portion solution comprises:
drying the shaped needle tip portion solution first time to allow the shaped needle tip portion solution to be initially solidified; and
pressing the shaped needle tip portion solution second time using a second needle tip portion pressing device to allow the shaped needle tip portion solution which is initially solidified to be further shaped; and
drying the shaped needle tip portion solution which is further shaped second time to obtain the needle tip portion.

18. The method of claim 14, wherein a weight percentage of the adhesive material in the adhesion layer solution is from 3% to 60% and a weight percentage of the viscosity-reducing substance in the adhesion layer solution is from 0.15% to 30% based on 100% by a total weight of the adhesion layer solution.

19. The method of claim 18, wherein a weight percentage of the viscosity-reducing substance in the adhesion layer solution is from 3% to 15% based on 100% by a total weight of the adhesion layer solution.

20. The method of claim 14, wherein the viscosity-reducing substance comprises sucrose, lactose, mannitol, mannose, galactose, glucose, fructose, maltose, trehalose, polyethylene glycol, or a combination thereof.

21. The method of claim 14, wherein the step of filling the adhesion layer solution in the microneedle mold which retains the needle tip portion comprises pressing the adhesion layer solution using an adhesion layer pressing device to allow shaping the adhesion layer solution to cover the needle tip portion.

22. The method of claim 14, wherein the step of providing the supporting portion comprises:
mixing a supporting portion material with water to obtain a supporting portion solution;
filling the supporting portion solution in a supporting portion mold to obtain a shaped supporting portion solution; and
drying the shaped supporting portion solution to obtain the supporting portion.

23. The method of claim 22,
wherein the step of filling the supporting portion solution in the supporting portion mold comprises:
covering the supporting portion mold with the supporting portion solution; and
pressing the supporting portion solution first time using a first supporting portion pressing device to allow the supporting portion solution to fill up the supporting portion mold and obtain the shaped supporting portion solution; and
wherein the step of drying the shaped supporting portion solution comprises:
drying the shaped supporting portion solution to allow the shaped supporting portion solution to be initially solidified;
pressing the shaped supporting portion solution second time using a second supporting portion pressing device to allow the shaped supporting portion solution which is initially solidified to be compacted on a bottom of the supporting portion mold;
covering the supporting portion mold with the supporting portion solution again;
pressing the supporting portion solution using the first supporting portion pressing device to allow the supporting portion solution covering the supporting portion mold again to fill up the supporting portion mold and cover the shaped supporting portion solution which is compacted and obtain a secondary shaped supporting portion solution; and
drying the secondary shaped supporting portion solution to allow the secondary shaped supporting portion solution to be shaped into the supporting portion.

24. The method of claim 14, wherein the step of providing the supporting portion, comprising:
disposing a supporting portion material on a supporting portion mold;
heating the supporting portion material to allow the supporting portion material to be melted and fill up the supporting portion mold; and
cooling the supporting portion material to obtain the supporting portion.
